(19) United States
(12) Patent Application Publication
(10) Pub. No.: US 2005/0142129 A1
(43) Pub. Date: Jun. 30, 2005

(54) Methods of preventing morbidity and mortality by perioperative administration of a blood clotting inhibitor

(76) Inventor: Dennis T. Mangano, Hillsborough, CA (US)

Correspondence Address: WILSON SONSINI GOODRICH & ROSATI, 650 PAGE MILL ROAD, PALO ALTO, CA, 943041050, US

(21) Appl. No.: 10/272,187

(22) Filed: Oct. 15, 2002

(51) Int. Cl. A61K 38/48; A61K 31/727; A61K 31/519; A61K 31/366; A61K 31/12

(52) U.S. Cl. 424/94.64; 514/2; 514/56; 514/165; 514/262.1; 514/457; 514/690

(57) ABSTRACT

The present invention provides methods of using a blood clotting inhibitor to reduce post-surgical morbidity and mortality. In particular, perioperative use of a blood clotting inhibitor decreases surgical complications without significant adverse effects and is cost effective.

### 1. FIELD OF THE INVENTION

The present invention relates to methods of preventing morbidity and mortality by administration of a blood clotting inhibitor perioperative to cardiac and noncardiac surgery, and continuing after hospital discharge for one year or more.

### 2. BACKGROUND OF THE INVENTION

Post-surgical complications are a significant source of morbidity and mortality, and healthcare expenditure.

For cardiac surgery, approximately one million patients undergo such every year, and approximately one in six develops a serious major organ complication relating to the heart, brain, kidney, GI tract and lung (Mangano, et al., 1997, J. Intensive Care Med. 12: 148-160). Yet despite numerous advances in monitoring and technique, no drug has been shown to reduce or prevent these complications. The preoccupation has been with bleeding, and drugs are now used to prevent such. However, drugs which inhibit bleeding generally cause thrombosis, and therefore may induce ischemia and irreversible organ injury (Cosgrove, et al., 1992, Ann. Thorac. Surg. 54: 1031-36).

For noncardiac surgery, approximately 250 million patients undergo such every year, and approximately four percent develop a serious major organ complication relating to the heart (Mangano, et al., 1990, Anesthesiology 2: 153-84; Mangano, et al., 1990 NEJM 323: 1781-88). Only one drug has been shown to mitigate injury-atenolol (Mangano, et al., 1996, NEJM 335: 1713-20). As well, concerns for bleeding predominate, and drugs preventing thrombosis (anti-platelet, anti-clotting) are virtually contraindicated (Eagle, et al., 1999, JACC 34: 1262-1347; Pearson, et al., 1994, Circulation 90: 3125-33; Baumgartner, et al., 1994, Johns Hopkins Manual of Surgical Care, Mosby Yearbook, St. Louis).

However, for both cardiac and noncardiac surgery, marked excitotoxic and inflammatory responses occur for days after surgery, if not months after surgery (Silicano and Mangano, 1990, Mechanisms and Therapies. In: Estafanous, ed. Opioids in Anesthesia Butterworth Publishers, pp. 164-178). Such markedly exaggerated responses are associated with platelet and clotting factor activation, which may precipitate thrombosis.

Although recognized as a possibility, such agents are relatively—and in some cases (fibrinolytics), absolutely—contra-indicated because of fear of excessive hemorrhage at the surgical site, as well as at other sites (Eagle, et al., 1999, JACC 34: 1262-1347; Pearson, et al., 1994, Circulation 90: 3125-3133; Baumgartner, et al., 1994, Johns Hopkins Manual of Surgical Care, Mosby Yearbook, St. Louis). Further, some believe—especially after cardiac surgery—that platelet and clotting factor function are depressed after surgery, so that thrombosis is not an issue (Kestin, et al., 1993, Blood 82: 107-117; Khuri, et al., 1992, J. Thorac. Cardiovasc. Surg. 104: 94-107). Thus, no effort has been made to investigate the use of anti-clotting agents immediately following surgery.

Finally, Applicants have shown that perioperative events manifest over six to eight months or longer (Mangano, et al. 1992, JAMA 268: 233-39); thus, continuation of use of such anti-clotting agents throughout the in-hospital, and then post-discharge course, is rational.

Surgery patients—now numbering 40 million per year in the U.S. alone—are aging nearly twice as rapidly as the overall population. (See, Mangano, et al., 1997, J. Intensive Care Med. 12: 148-160).

The current standards of care are unsatisfactory to address this critical problem, and novel approaches are desperately needed to prevent post-surgical complications in our aging population.

### 3. SUMMARY OF THE INVENTION

Accordingly, the present invention provides methods of preventing or reducing post-surgical morbidity and mortality. Significantly, the prevention or reduction of post-surgical morbidity and mortality can extend beyond hospitalization. In certain aspects, the methods comprise the perioperative and long-term administration of a blood clotting inhibitor to prevent or reduce post-surgical complications. The blood clotting inhibitor can be administered perioperatively, that is, prior to, during and/or after surgery and after hospital discharge, for example, six months, one year or longer.

The invention is based, in part, on Applicant's surprising discovery that perioperative administration of a blood clotting inhibitor can significantly reduce post-surgical morbidity and mortality. The invention can provide a reduction in post-surgical morbidity and mortality during the post-surgical hospitalization recovery period, and even over the years after discharge from hospital. Administration of a blood clotting inhibitor can reduce the number and severity of post-surgical and long-term adverse events. In addition, perioperative use of a blood clotting inhibitor need not increase perioperative or post-surgical complications, such as bleeding and can be extremely cost effective.

In one embodiment, the blood clotting inhibitor can be any drug, agent or pharmaceutical composition that blocks, prevents or inhibits the formation of thrombosis (blood clots), or that dissolves or breaks down a blood clot. The blood clotting inhibitor can be any blood clotting inhibitor currently known to those of skill in the art or one later developed. The blood clotting inhibitor can be from any drug class known to those of skill in the art including, but not limited to, antiplatelet agents, thrombolytic enzymes, aggregation inhibitors, glycoprotein IIb/IIIa inhibitors, glycosaminoglycans, thrombin inhibitors, anticoagulants, heparin, low molecular weight heparins, coumarins, indandione derivatives and tissue plasminogen activators and combinations thereof.

The blood clotting inhibitor can be given perioperatively and long term. Perioperative administration includes the time period before surgery, after surgery, during surgery, and/or any combination as described herein. For example, the blood clotting inhibitor can be administered 6 months, 3 months, 1 month, 1 week, 96 hours, 48 hours or less, perioperatively; that is, the blood clotting inhibitor can be administered 6 months, 3 months, 1 month, 1 week, 96 hours, 48 hours or less before surgery, 6 months, 3 months, 1 month, 1 week, 96 hours, 48 hours or less after surgery, or both 6 months, 3 months, 1 month, 1 week, 96 hours, 48 hours or less before and after surgery. In one embodiment, the blood clotting inhibitor can be administered 6 days, 5 days, 4 days, 3 days, 2 days and/or 1 day perioperatively. In another embodiment, the blood clotting inhibitor can be administered 8, 6, 4, 2 or 1 hour perioperatively. The perioperative administration of a blood clotting inhibitor if given both preoperatively and postoperatively need not be given an equal number of hours preoperatively and postoperatively. For example, a blood clotting inhibitor can be administered within 1 week prior to surgery and 6 hours after surgery. The blood clotting inhibitor can be given during surgery. For example, the blood clotting inhibitor can be given contemporaneously with the use or discontinuation of cardiopulmonary bypass or contemporaneously with reperfusion of an ischemic area (e.g., for cardiac surgery), and immediately following wound closure (e.g., noncardiac surgery).

Long term administration includes the time period immediately after surgery, through discharge, and during the months thereafter, even continuing for two years—the time interval over which the dramatic effects of surgery can occur. For example, the blood clotting inhibitor can be continued from the perioperative period to long term using an oral or transdermal formulation, with dose adjustments made, as needed, by the primary care physician. Or, the patient may be started on the blood clotting inhibitor on discharge and then followed by a physician. Finally, differing formulations may be given with differing schedules for those patients who may have had a complicated postoperative course.

The blood clotting inhibitor can be given in any dose known to those of skill in the art. The dose of blood clotting inhibitor can be sub- or supra-therapeutic. The route of administration of the blood clotting inhibitor can be any route known to those of skill in the art. Such routes of administration include, but are not limited to, oral, parenteral and transdermal.

In one embodiment, the surgery can be cardiac surgery. In certain embodiments, the surgery can be non-cardiac surgery or ambulatory surgery. The surgery can be for example, abdominal, neurological, gynecological, orthopedic, urological, vascular and surgery related to otolaryngology.

Blood clotting inhibitors are generally discontinued prior to surgery for fear of excessive hemorrhage. In most instances when patients on long term anti-coagulant therapy are scheduled for surgery, typically, the anti-coagulant is discontinued for approximately 14 days prior to surgery. In addition, in such cases, the anti-coagulant therapy is often restarted only after hospital discharge. In many cases, blood clotting inhibitors are absolutely contraindicated perioperatively. See, e.g., Eagle, et al., 1999, JACC 34: 1262-1347; Pearson, et al., 1994, Circulation 90: 3125-3133; Baumgartner, et al., 1994, Johns Hopkins Manual of Surgical Care, Mosby Yearbook, St. Louis. Although traditional surgical practice is loathe to use a blood clotting inhibitor in the perioperative arena, Applicant's use of a perioperative blood clotting inhibitor significantly reduces post-surgical morbidity and mortality as demonstrated in the example herein. Patients receiving a perioperative blood clot inhibitor had reductions in post-surgical complications of a thromboembolic origin, such as, myocardial infarction, stroke, transient ischemic attacks, renal failure, renal insufficiency and bowel infarction. Surprisingly, patients treated perioperatively with a blood clotting inhibitor also experienced less adverse effects related to bleeding.

### 4. BRIEF DESCRIPTION OF THE FIGURES

FIG. 1 provides fatal (N=164) and non-fatal (N=748) ischemic outcomes among the aspirin and non-aspirin groups;

FIG. 2 provides thirty-day survival by aspirin use among the 5065 study patients;

FIG. 3A provides mortality associated with platelet transfusion among the aspirin and non-aspirin groups; and

FIG. 3B provides mortality associated with use of anti-fibinolytic therapy among the aspirin and non-aspirin groups.

### 5. DETAILED DESCRIPTION OF THE INVENTION

5.1 Definitions

“Administered” or “administration” refers to the introduction of the blood clotting inhibitor to the patient. Administration refers to the giving of a dose by a person, including, for example, a health care provider or the patient himself.

“Blood clotting inhibitor” refers to any drug, agent or pharmaceutical composition that can block, prevent or inhibit the formation of blood clots or dissolves or breaks down a blood clot. A blood clotting inhibitor can be any blood clotting inhibitor currently known to those of skill in the art or one later developed. The blood clotting inhibitor can be from any drug class of blood clotting inhibitors known to those of skill in the art including, but not limited to, antiplatelet agents, thrombolytic enzymes, aggregation inhibitors, glycoprotein IIb/IIIa inhibitors, glycosaminoglycans, thrombin inhibitors, anticoagulants, heparin, low molecular weight heparins, coumarins, indandione derivatives, tissue plasminogen activators and combinations thereof. The blood clotting inhibitors can be in any pharmaceutical dosage form and administered by any route known to those of skill in the art.

“Perioperative” refers to the time period before surgery (pre-operative), after surgery (post-operative), during surgery (intra-operative), and/or any combination as described herein. For example, the blood clotting inhibitor can be administered 48 hours perioperatively; that is, the blood clotting inhibitor can be administered 48 hours before surgery (pre-operatively), 48 hours after surgery (post-operative), during surgery (intra-operative) or any combination of these administration times. The administration during the perioperative period can be a single dose or multiple doses within the perioperative time period. It will be appreciated by those of skill in the art that ‘pre-operative’ refers to the time period before surgery, ‘post-operative’ refers to the time period after surgery and ‘intra-operative’ refers to the time period during surgery.

“Long-term” refers to the time period after hospital discharge, and extending for 6 months or longer. For example, the blood clotting inhibitor can be administered at the time of discharge as one dose, and then may be continued for 6 months, one year or longer, after the perioperative period.

“Surgery” or “surgical” refers to any manual or operative methods or manipulations for the treatment or prevention of disease, injury or deformity. Surgery includes methods or manipulations conducted while a patient is under anesthesia, including local or general anesthesia. Surgery can be performed by a doctor, surgeon or dentist, generally in a hospital or other health care facility. Patients undergoing surgery can be hospitalized or ambulatory, e.g., out-patient surgery. Surgery does not include percutaneous intervention (PTI) or percutaneous transluminal coronary angioplasty (PTCA).

“Coronary artery bypass graft” or “CABG” refers to cardiac surgery wherein one or more bypass grafts are implanted between the aorta and the coronary blood vessel, commonly using saphenous veins or internal mammary arteries as grafts. “Vein graft CABG” refers to CABG surgery wherein a saphenous vein(s) is used for grafting. “Artery graft CABG” refers to CABG surgery wherein an internal mammary artery (arteries) is used for grafting.

5.2 Timing of Administration

The blood clotting inhibitor can be administered perioperatively; that is, before surgery, after surgery and/or during surgery, or any combination as described herein. For example, if the half-life of the drug is long (24-48 hours), the blood clotting inhibitor can be administered as one dose within 48 (or 24) hours prior to surgery with repeated doses during or after surgery. Drugs with shorter half-lives can be given sooner before surgery and then be administered during or after surgery. In some patients, and some circumstances, the treating physician may decide to suspend preoperative treatment, and only start administration post-operatively, e.g., 48 hours after surgery, after wound closure to assure that no bleeding has occurred in the field (no open blood vessels) before starting anti-clotting therapy. Such immediate postoperative administration of a blood clotting inhibitor is within the scope of the invention.

Perioperative administration includes the time period before surgery (pre-operative), after surgery (post-operative), during surgery (intra-operative), and/or any combination as described herein. For example, the blood clotting inhibitor can be administered 6 months, 3 months, 1 month, 1 week, 96 hours, 48 hours or less perioperatively; that is, the blood clotting inhibitor can be administered 6 months, 3 months, 1 month, 1 week, 96 hours, 48 hours or less before surgery, 6 months, 3 months, 1 month, 1 week, 96 hours, 48 hours or less after surgery, or both 6 months, 3 months, 1 month, 1 week, 96 hours, 48 hours or less before and after surgery. In addition, the blood clotting inhibitor can be administered, for example, 36, 24, 12, 8, 6, 4, 2 or 1 hour perioperatively; that is the blood clotting inhibitor can be administered, for example, 36, 24, 12, 8, 6, 4, 2 or 1 hour before surgery and/or 36, 24, 12, 8, 6, 4, 2 or 1 hour after surgery and/or during surgery. One can administer the blood clotting inhibitor for an equal number of hours pre and post surgery. For example, one can administer the blood clotting inhibitor 48 hours prior to surgery and 48 hours after surgery. One can administer the blood clotting inhibitor for an unequal number of hours pre and post surgery. For example, one can administer the blood clotting inhibitor 48 hours prior to surgery and 24 hours after surgery. One can administer the blood clotting inhibitor, for example, 36 hours prior to surgery and 36 hours after surgery. One can administer the blood clotting inhibitor 36 hours prior to surgery and 12 hours after surgery. One can administer the blood clotting inhibitor, for example, 12 hours prior to surgery and 12 hours after surgery. One can administer the blood clotting inhibitor, for example, 8 hours prior to surgery and 8 hours after surgery. One can administer the blood clotting inhibitor, for example, 6 hours prior to surgery and 8 hours after surgery. One can administer the blood clotting inhibitor, for example, 6 hours prior to surgery and 6 hours after surgery. One can administer the blood clotting inhibitor, for example, 8 hours prior to surgery and 4 hours after surgery. One can administer the blood clotting inhibitor 4 hours prior to surgery and 4 hours after surgery. One can administer the blood clotting inhibitor 2 hours prior to surgery and 8 hours after surgery. One can administer the blood clotting inhibitor 4 hours prior to surgery and 1 hour after surgery. One can administer the blood clotting inhibitor, for example, 24 hours prior to surgery and during surgery. One can administer the blood clotting inhibitor, for example, during surgery and 6 hours after surgery.

Administration in the perioperative period can be a single, one time dose or multiple doses of the blood clotting inhibitor. In certain embodiments, perioperative administration can be continuous, uninterrupted administration of the blood clotting inhibitor (e.g. a continuous infusion or transdermal delivery). In another embodiment, perioperative administration is single or multiple discreet administration(s) within the perioperative time frame (e.g. a single dose given within the perioperative period or multiple doses given within the perioperative period). In one embodiment, the blood clotting inhibitor can be administered within 6 days, 5 days, 4 days, 3 days, 2 days or 1 day perioperatively. In another embodiment, the blood clotting inhibitor can be administered within 48 hours, 36 hours, 24 hours, 12 hours, 8 hours, 6 hours or 1 hour perioperatively.

The blood clotting inhibitor can be administered during surgery, for example, contemporaneously with the use or discontinuation of cardiopulmonary bypass or contemporaneously with reperfusion of an ischemic area. Administration can be continued long term for example, after surgery, following discharge from hospital and for six months, one year or longer post-operatively.

In certain embodiments, when the patient is on chronic blood clottting inhibitor therapy prior to surgery, the blood clotting inhibitor is not discontinued pre-operatively, in contrast to standard practice.

Perioperatively, the patient need not be conscious for administration of the blood clotting inhibitor. For example, the blood clotting inhibitor can be given during surgery while the patient is under anesthesia. During some ambulatory or outpatient surgeries, the patient remains conscious and in such a situation, the blood clotting inhibitor can be given during surgery when the patient is conscious.

Such therapy can be continued after discharge. In the course of long-term treatment, as described above, the formulation and dosage can be continued or adjusted, or the type of blood clotting inhibitor can be changed to another blood clotting inhibitor.

5.3 Surgery and Surgical Complications

The present invention provides methods of preventing or reducing post-surgical morbidity and mortality. In certain aspects the methods comprise the perioperative administration of a blood clotting inhibitor to prevent or reduce post-surgical complications. The blood clotting inhibitor can be administered perioperatively; that is prior to, during and/or after surgery, and after hospital discharge. Significantly, the prevention or reduction of post-surgical morbidity and mortality extends beyond hospitalization.

Surgery refers to any manual or operative methods or manipulations for the treatment or prevention of disease, injury or deformity. Surgery includes methods conducted while a patient is under anesthesia, including local or general anesthesia. Surgery can be performed by a doctor, surgeon or dentist, generally in a hospital or other health care facility. Patients undergoing surgery can be hospitalized or ambulatory, e.g., out-patient surgery. For purposes of this invention surgery includes, but is not limited to: abdominal surgery (e.g. surgery of the abdominal viscera), bench surgery (e.g. surgery performed on an organ that has been removed from the body, after which it can be reimplanted), cardiac (e.g. surgery of the heart), cerebral (e.g. surgery upon the brain), cineplastic (e.g. surgery to create a tunnel through a muscle adjacent to the stump of an amputated limb, to permit use of the muscle in operating a prosthesis), cosmetic (e.g. surgery to improve a patient's appearance by plastic restoration, correction or removal of blemishes), dentofacial (e.g. surgery involving defects of the face and structures of the mouth), neurological (e.g. surgery involving the peripheral or central nervous system), oral (e.g. surgery involving defects of the mouth, jaws and associated structures), orthopedic (e.g. surgery dealing with bones and bony structures), pelvic (e.g. surgery involving the pelvis, predominately obstetrical and gynecological), plastic (e.g. surgery involving the restoration, reconstruction, correction or improvement in the shape and appearance of body structures that are defective, damaged or misshapened by injury, disease, or growth and development) or rectal (e.g. surgery of the rectum), urological (e.g. surgery related to the genitourinary system, predominately in males), vascular (e.g. surgery of the blood vessels), and surgery related to otolaryngology (e.g. surgery of the ears, nose, throat or related structures). The surgery can be conservative (e.g. surgery to preserve or remove with minimal risk, diseased or injured organs, tissues, or extremities) or radical (e.g. surgery designed to extirpate all areas of locally extensive disease and adjacent zones of lymphatic drainage). In certain embodiments, the surgery can be cardiac surgery, including cardiac valve replacement, heart and heart-lung transplant, and implantation of artificial heart devices and defibrillators, valve replacement or valve repair and congenital surgery.

In certain embodiments, when the cardiac surgery is CABG, the surgery can be coronary artery bypass grafting using saphenous veins or internal mammary arteries, referred to herein as vein graft CABG or artery graft CABG, respectively. In one embodiment, when the surgery is vein graft CABG, the blood clotting inhibitor is not aspirin administered from the time beginning 12 hours pre-operatively through seven hours post-operatively. In another embodiment, when the surgery is vein graft CABG, the blood clotting inhibitor is not dipyridamole administered from the time beginning 48 hours pre-operatively through 24 hours post-operatively. See, Goldman, et al., 1988, Circulation 77: 1324-32; Chesebro, et al., 1982, NEJM 307: 73-8; Chesebro, et al., 1984, NEJM 310: 209-14. In another embodiment, when the surgery is vein graft CABG, the blood clotting inhibitor is not ticlopidine or aprotinin. See, Drug Facts and Comparisons, updated monthly, September, 2002, Facts and Comparisons, Wolters Kluwer Company, St. Louis, Mo.

In certain embodiments, when the cardiac surgery is artery graft CABG, the blood clotting inhibitor is not aprotinin.

The invention can be used on a wide variety of surgeries, including, but not limited to, cardiac, abdominal, neurological, gynecological, orthopedic, urological, vascular, and surgery related to otolaryngology. More specifically, surgery includes, small and large bowel resection, appendectomy, laparoscopy, paracentesis, transurethral resection of the prostate (TURP), hysterectomy, tubal ligation, vasectomy, salpingo-oophorectomy, Cesarean section, hemorrhoidectomy, tonsillectomy, myringodectomy, placement of myringotomy tubes, removal of polyp(s) from the colon and rectum, repair of rectal prolapse, removal and treatment of neoplasms of the bowel, curettage, thoracentesis, thoracotomy, rhinoplasty, liposuction and the like.

Ambulatory or outpatient surgery includes surgery for which hospitalization and/or general anesthesia is generally not required. Such surgeries include placement of myringotomy tubes, hemorrhoidectomy and the like.

The invention can reduce post-surgical morbidity and mortality during the post-surgical hospitalization recovery period and after discharge from hospital. The post-surgical morbidity and mortality can be from any surgical complication. Complications of surgery can be cardiac (myocardial infarction, congestive heart failure, serious cardiac dysrhythmias, ischemia) neurological (stroke, encephalopathy, cognitive dysfunction, transient ischemic attacks, seizures), renal (failure, dysfunction or renal death), gastrointestinal (infarction, ileus, ischemia, mesenteric thrombosis or GI death), pulmonary (failure, respiratory distress syndrome, edema), and the like.

5.4 Blood Clotting Inhibitor

The present invention provides methods of preventing or reducing post-surgical morbidity and mortality. In certain aspects the methods comprise the perioperative administration of a blood clotting inhibitor to prevent or reduce post-surgical complications. The blood clotting inhibitor can be administered perioperatively; that is prior to, during and/or after surgery, and after hospital discharge.

The blood clotting inhibitor of the present invention can be any drug, agent or pharmaceutical composition that prevents or inhibits blood clotting. The inhibitor can act by preventing or inhibiting blood clot formation by any of a variety of mechanisms including reduction of blood clotting factors or reducing platelet activation or aggregation, or mitigating the effects of instigating factors, such as inflammation or stress. The blood clotting inhibitor can also act by breaking down or dissolving a blood clot after formation. It will be apparent to those of skill in the art that there are several classes of blood clotting inhibitor, including antiplatelet agents, thrombolytic enzymes, aggregation inhibitors, glycoprotein IIb/IIIa inhibitors, glycosaminoglycans, thrombin inhibitors, anticoagulants, heparins, low molecular weight heparins, coumarins, indandione derivatives and tissue plasminogen activators. See, The Physicians' Desk Reference (56^{th} ed., 2002) Medical Economics; Mosby's Drug Consult, 2002, Elsevier Science; Goodman and Gilman's The Pharmacologic Basis of Therapeutics, (9^{th} ed. 1996) Pergamon Press; Drug Facts and Comparisons, updated monthly, September, 2002, Facts and Comparisons, Wolters Kluwer Company, St. Louis, Mo.

For the purposes of this invention, any drug, agent or pharmaceutical composition that prevents or inhibits the formation of blood clots or dissolves or breaks down a blood clot is suitable for use in the present invention. Such a blood clotting inhibitor can be, for example, cilostazol (PLETAL®, Otsuka), clopidogrel (PLAVIX®, Sanofi), ticlopidine (TICLID®, Syntex), tirofiban (AGGRASTAT®, Merck), eptifibatide (INTEGRILIN®, COR Therapeutics), abciximab (REOPRO®, Eli Lilly), anagrelide (AGRYLIN®, Roberts), dipyridamole (PERSANTINE®, Boehringer Ingelheim), aspirin (ECOTRIN®, and others), dipyridamole/aspirin (AGGRENOX®, Boehringer Ingelheim), dalteparin (FRAGMIN®, Pharmacia), enoxaparin (LOVENOX®, Aventis), tinzaparin (INNOHEP®, DuPont), heparin (various), danaparoid (ORGANON®, Organon), antithrombin III (THROMBATE®, Bayer), lepirudin (REFLUDAN®, Hoechst-Marion Roussel), argatroban (ACOVA®, SmithKlineBeecham), bivalirudin (ANGIOMAX®, Medicines Company), warfarin (COUMADIN®, DuPont) anisidione (MIRADON®, Schering), alteplase (ACTIVASE®, Genetech), reteplase (RETAVASE®, Boehringer Mannheim), tenecteplase (TNKASE®, Genentech), drotrecogin (XIGRIS®, Eli Lilly), anistreplase (EMINASE®, Roberts), streptokinase (STREPTASE®, Astra), urokinase (ABBOKINASE®, Abbott) and combinations thereof.

It will be appreciated by those of skill in the art that blood clotting inhibitors are used for the treatment of occluded catheters and for the maintenance of patency of vascular access devices. Heparin, urokinase, streptokinase and alteplace are generally employed for such uses. The use of blood clotting inhibitors for the treatment of occluded catheters and for the maintenance of patency of vascular access devices is not within the scope of the invention.

In certain embodiments where the blood clotting inhibitor is a low molecular weight heparin, the surgery is preferably not hip replacement, knee replacement or abdominal surgery. When the drug is dalteparin, the dose is preferably not 2500 IU subcutaneously once daily, starting 1 to 2 hours preoperatively and repeating once daily for 5-10 post-operatively or 5000 IU subcutaneously the evening before surgery and repeated once daily for 5-10 days postoperatively. When the drug is enoxaparin, the dose is preferably not 40 mg once daily subcutaneously given initially 9 to 15 hours prior to surgery and continued for 21 days or 40 mg once daily subcutaneously starting 2 hours prior to surgery and continued for 7 to 10 days; 12 days if tolerated.

In certain embodiments where the blood clotting inhibitor is heparin, the surgery is preferably not abdominothoracic or cardiac surgery. When the drug is heparin, the dose is preferably not 5000 Units subcutaneously 2 hours before surgery and 5000 Units every 8 to 12 hours thereafter for 7 days or until the patient is fully ambulatory. When the drug is heparin, the dose is preferably not 150 Units/kg for patients undergoing total body perfusion for open heart surgery. When the drug is heparin, the dose is preferably not 300 Units/kg for procedures less than 60 minutes or 400 Units/kg for procedures longer than 60 minutes.

In certain embodiments where the blood clotting inhibitor is danaparoid, the surgery is not elective hip replacement surgery. When the drug is danaparoid, the dose is preferably not 750 anit-Xa units twice daily subcutaneously beginning 1 to 4 hours preoperatively and then not sooner than 2 hours after surgery continued for 7-10 days postoperatively.

In certain embodiments where the blood clotting inhibitor is warfarin, the surgery is preferably not cardiac valve replacement surgery. When the drug is warfarin, the dose is preferably not 1 mg daily, up to 20 days preoperatively.

In certain embodiments, when the cardiac surgery is vein graft CABG, the blood clotting inhibitor is not aspirin administered within 12 hours pre-operatively through seven hours post-operatively. In certain embodiments, when the cardiac surgery is vein graft CABG, the blood clotting inhibitor is not dipyridamole administered within 48 hours pre-operatively through 24 hours post-operatively. See, Goldman, et al., 1988, Circulation 77: 1324-32; Chesebro, et al., 1982, NEJM 307: 73-8; Chesebro, et al., 1984, NEJM 310: 209-14. In certain other embodiments, when the cardiac surgery is vein graft CABG, the blood clotting inhibitor is not ticlopidine or aprotinin. See, Drug Facts and Comparisons, updated monthly, September, 2002, Facts and Comparisons, Wolters Kluwer Company, St. Louis, Mo.

Aprotinin is indicated for CABC surgery in one of two dosing regimens, regimen A or regimen B. Regimen A is administration of a 2 million KIU (kallikrein inhibitor units) intravenous loading dose; 2 million KIU into the cardiopulmonary bypass machine (known as pump prime volume) and 500,000 KIU/hr of operation time as a continuous maintenance intravenous infusion. Regimen B is administration of a 1 million KIU intravenous loading dose, 1 million KIU into the pump prime volume and 250,000 KIU/hr of operation time as a continuous maintenance intravenous infusion. Administration of aprotinin begins after anesthetic induction but prior to sternotomy and is continued until surgery is complete and the patient leaves the operating room. Drug Facts and Comparisons, updated monthly, September, 2002, Facts and Comparisons, Wolters Kluwer Company, St. Louis, Mo. In certain embodiments when the surgery is vein graft or artery graft CABG, the blood clotting inhibitor is not aprotinin.

The blood clotting inhibitor can be a combination of two or more blood clotting inhibitors. Combinations of blood clotting inhibitors can include blood clotting inhibitors from more than one drug class as described herein. In addition, the combination of blood clotting inhibitors can include different routes of administration for each blood clotting inhibitor. The combination of blood clotting inhibitors can be administered simultaneously or contemporaneously. In addition, the combination of blood clotting inhibitors can be administered separately.

5.5 Dosage, Formulation and Administration

The blood clotting inhibitor described herein, can be administered into a patient for the reduction of mortality and morbidity following surgery by any means that produces contact of the blood clotting inhibitor with the blood clotting inhibitor's site of action in the body of the patient. The blood clotting inhibitor can be a pharmaceutical composition that can be administered by any means available. It will be apparent to those of skill in the art that a pharmaceutical composition can be generally administered with a pharmaceutical carrier. The pharmaceutical composition and/or pharmaceutical carrier can be selected on the basis of the chosen route of administration and standard pharmaceutical practice. The pharmaceutical compositions of the invention can be adapted for oral, parenteral or topical administration, and can be in unit dosage form, in a manner well known to those skilled in the pharmaceutical art. Parenteral administration includes, but is not limited to, injection subcutaneously, intravenously, intraperitoneally or intramuscularly. It will be apparent to one of skill in the art that, for example, oral dosage forms can be administered by a number of routes, including, but not limited to rectal and vaginal and via any means to deliver substance to the gastrointestinal tract, such as via a nasogastric tube.

The dose administered will, of course, vary depending upon known factors, such as: the pharmacodynamic characteristics of the particular blood clotting inhibitor and its mode and route of administration; the age, health, height and weight of the patient; the kind of concurrent treatment(s); the frequency of treatment(s); and the effect desired. The dose of the blood clotting inhibitor need not remain constant but can be adjusted according to parameters that are well known to those of skill in the art. In addition, the dose of blood clotting inhibitor can be sub- or supra-therapeutic.

A single dose of active ingredient can be within the normal dosage range appropriate for the individual patient. For instance, aspirin can be used orally at 40 mg-160 mg/day. Dipyridamole can be used at orally at 75 mg-100 mg four times daily. Aspirin and dipyridamole can be given in combination as a single commercially available product at a dose of 25 mg aspirin/200 mg dipyridamole (AGGRENOX®) or the compositions can be given together contemporaneously as individual compositions in the dosage rages described herein. Heparin can be used subcutaneously with an initial dose of 10,00-20,000 Units (which can be preceded by an intravenous loading dose of 5,000 units), followed by 8,000-10,000 units every 8 hours or 15,000 to 20,000 units ever 12 hours, adjusting for partial thromboplastin time (PTT) to about 1.5 to 2 times normal. Warfarin can be used orally or parenterally at 0.5-30 mg/day. Cilostazol can be used orally at 50-100 mg twice daily. Clopidogrel can be used orally at 75 mg once daily, with or without a 300 mg loading dose. Ticlopidine can be used orally at 250 mg twice daily. Tirofiban can be used parenterally at 0.4 mcg/kg/min over 30 minutes, then continued at 0.1 mcg/kg/min. Eptifibatide can be used parenterally at 180 mcg/kg as an intravenous bolus, followed by 2 mcg/kg/min continuous infusion with a second bolus, given 10 minutes after the initial intravenous bolus. The second parenteral bolus dose can be 180 mcg/kg. Abciximab can be used parenterally at 0.25 mg/kg infused over 10 to 60 minutes as an intravenous bolus, followed by continuous infusion of 0.125 mcg/kg/min, to a maximum of 10 mcg/min, for 12 hours. Anagrelide can be used orally at 0.5 mg four times daily to 1 mg twice daily titrated up to a maximum of 10 mg/day. Dalteparin can be used subcutaneously at 2500-5000 IU once to twice daily. Enoxaparin can be used subcutaneously at 1 mg/kg once to twice daily. Tinzaparin can be used subcutaneously at 175 anti-Xa IU/kg once daily. Danaparoid can be used subcutaneously at 750 anti-Xa units twice daily. Antithrombin III can be used parenterally at a dose based on the pretherapy plasma antithrombin m (AT) level. Dosage can be calculated by:

$$Units required ( IU ) = \frac{[ \text{desired-baseline} ( AT ) \text{level} ]}{1.4} \times \text{weight} ( kg )$$

or alternatively:

$$\begin{matrix}Number of & & body & & Desired & & Reciprocal \\ Factor IX & = & weight & \times & Factor IX & \times & Of observed \\ Required & & ( kg ) & & Increase & & Recovery \\ ( IU ) & & & & ( \% or IU \text{/} d L ) & & ( IU \text{/} kg per IU \text{/} dL )\end{matrix}$$

(See, Drug Facts and Comparisons, updated monthly, September, 2002, Facts and Comparisons, Wolters Kluwer Company, St. Louis, Mo.).

Lepirudin can be given parenterally in a bolus dose of 0.4 mg/kg, intravenous push over 15-20 seconds, followed by 0.15 mg/kg continuous intravenous infusion. Argatroban can be given at 2 mcg/kg/min as a continuous infusion. Bivalirudin can be given at 1 mg/kg intravenous bolus followed by a 4 hour intravenous infusion at 2.5 mg/kg/hr. Anisidione can be used orally at 25-300 mg/day. Alteplase can be given intravenously in patients weighing more than 67 kg, at a dose of 100 mg administered as a 15 mg intravenous bolus, followed by 50 mg infused over the next 30 minutes and then 35 mg infused over the next 60 minutes. In patients weighing less than 67 kg, alteplase can be administered intravenously as a 100 mg total dose; a 15 mg intravenous bolus followed by 0.75 mg/kg infused over the next 30 minutes not to exceed 50 mg and then 0.5 mg/kg over the next 60 minutes, not to exceed 35 mg. Reteplase can be used parenterally as a 10 Unit intravenous bolus injection over 2 minutes, followed 30 minutes later by a second 10 Unit intravenous bolus injection over 2 minutes. Tenecteplase can be used parenterally at a dose of 30-50 mg, based on patient weight, and administered as a single bolus over 5 seconds. Drotrecogin can be used parenterally at 24 mcg/kg/hr for a total infusion duration of 96 hours. Anistreplase can be used parenterally at 30 Units administered intravenous over 2 to 5 minutes. Streptokinase can be used parenterally at a dose of 250,000 Units infused over 30 minute. In addition, streptokinase can be used intravenously at 20,000 IU bolus followed by a dose of 2,000 IU/minute for 60 minutes. Urokinase can be used parenterally at a dose of 4400 Units/kg over 10 minutes, followed by continuous infusion of 4400 Units/kg/hr at a rate of 15 ml/hr for 12 hours.

The active ingredient of a blood clotting inhibitor can be administered orally in solid or semi-solid dosage forms, such as hard or soft-gelatin capsules, tablets, or powders, or in liquid dosage forms, such as elixirs, syrups, or suspensions. It can also be administered parenterally, in sterile liquid dosage forms. Other dosage forms are potentially possible such as patches or ointment or transdermal administration.

Parenteral dosage forms can be, for example, injectable preparations including sterile suspensions, solutions or emulsions of the active ingredient in aqueous or oily vehicles. The compositions may also comprise formulating agents, such as suspending, stabilizing and/or dispersing agent. The formulations for injection may be presented in unit dosage form, e.g., in ampules or in multidose containers, and may comprise added preservatives.

An injectable formulation can be in powder form for reconstitution with a suitable vehicle, including but not limited to sterile pyrogen free water, buffer, dextrose solution, etc., before use.

For administration during surgery, the active ingredient can be administered directly into the cardiopulmonary bypass machine, directly into the pericardium or directly into the vessels exposed in the surgical field.

For prolonged delivery, the active ingredient can be formulated as a depot preparation, for administration by implantation; e.g., subcutaneous, intradermal, or intramuscular injection. Thus, for example, the active ingredient may be formulated with suitable polymeric or hydrophobic materials (e.g., as an emulsion in an acceptable oil) or ion exchange resins, or as sparingly soluble derivatives.

Alternatively, transdermal delivery systems manufactured as an adhesive disc or patch that slowly releases the active ingredient for percutaneous absorption may be used. To this end, permeation enhancers may be used to facilitate transdermal penetration of the blood clotting inhibitor.

For oral administration, the pharmaceutical formulations or the blood clotting inhibitor may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid.).

The preparations may also comprise buffer salts, flavoring, coloring and sweetening agents as appropriate. Preparations for oral administration may be suitably formulated to give controlled release of the active compound.

For buccal administration, the compositions may take the form of tablets or lozenges formulated in conventional manner. For rectal and vaginal routes of administration, the active ingredient may be formulated as solutions (for retention enemas) suppositories or ointments.

For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated comprising a powder mix cf the compound and a suitable powder base such as lactose or starch.

The compositions may, if desired, be presented in a pack or dispenser device that may comprise one or more unit dosage forms comprising the active ingredient. The pack may for example comprise metal or plastic foil, such as a blister pack. The pack or dispenser device may be accompanied by instructions for administration.

The blood clotting inhibitor can be administered by any suitable route known to those of skill in the art that ensures bioavailability in the circulation. Administration can be achieved by parenteral routes of administration, including, but not limited to, intravenous (IV), intramuscular (IM), intradermal, subcutaneous (SC), and intraperitoneal (IP) injections. In certain embodiments, administration is by a bypass machine, perfuser, infiltrator or catheter. In certain embodiments, the blood clotting inhibitor is administered by injection, by a subcutaneously implantable pump or by a depot preparation, in doses that achieve a therapeutic effect. Suitable dosage forms are further described in Remington's Pharmaceutical Sciences, 1990, 17th ed., Mack Publishing Company, Easton, Pa., a standard reference text in this field, which is incorporated herein by reference in its entirety.

Administration can be achieved through a variety of different treatment regimens. For example, several oral doses can be administered periodically during a single day, with the cumulative total of blood clotting inhibitor not reaching the daily toxic dose. Alternatively, the blood clotting inhibitor can be administered daily beginning, for example, 48 hours prior to surgery and continuing daily, for example, until 48 hours after surgery.

Intravenous injections can be administered periodically during a single day, with the cumulative total volume of the injections not reaching the daily toxic dose. Alternatively, one intravenous injection can be administered, for example, daily beginning, for example, 48 hours prior to surgery and continuing daily, for example, until 48 hours after surgery. The dose of the blood clotting inhibitor can vary. For example, an escalating dose can be administered. Depending on the needs of the patient, administration can be by slow infusion with a duration of more than one hour, by rapid infusion of one hour or less, or by a single bolus injection.

Other routes of administration may be used. For example, absorption through the gastrointestinal tract can be accomplished by oral routes of administration (including but not limited to ingestion, via nasogastric tube, buccal and sublingual routes). Alternatively, administration via mucosal tissue such as vaginal and rectal modes of administration can be utilized. In yet another alternative, the formulations of the invention can be administered transcutaneously (e.g., transdermally), or by inhalation. It will be appreciated that the preferred route may vary with the condition and age of the recipient.

The actual dose of blood clotting inhibitor will vary with the route of administration. The blood clotting inhibitor will generally be used in an amount effective to achieve the intended purpose. Of course, it is to be understood that the amount used will depend on the particular application.

The effective amount, for example, may vary depending on the type of surgery, condition of the patient, age of the patient, patient's weight, medical history of the patient, the manner of administration and the judgment of the prescribing physician. It will be appreciated by one of skill in the art that the degree of blood anticoagulation can be monitored by laboratory values such as prothrombin time (PT) and partial thromboplastin time (PTT). Determination of an effective amount is well within the capabilities of those skilled in the art, especially in light of the detailed disclosure provided herein.

The administration of a blood clotting inhibitor may be repeated intermittently. The blood clotting inhibitor can be administered alone or in combination with other drugs, for example, other presurgical drugs such as antibiotics or anesthetics.

### 6. EXAMPLE

### Administration of Aspirin Reduces Morbidity and Mortality Following Cardiac Surgery

The following example describes specific aspects of the invention to illustrate the invention and provide a description of the methods used to reduce morbidity and mortality following cardiac surgery. The example should not be construed as limiting the invention, as the example merely provides specific methodology useful in understanding and practicing the invention.

6.1 Materials and Methods

6.1.1. Patient Population and Methods

A prospective, longitudinal study enrolled 5,436 patients. Eligible patients included those with medically-refractory coronary artery disease and scheduled for coronary artery bypass surgery at 70 medical institutions among 17 countries in North and South America, Europe, the Middle East and Asia. At each institution, 100 patients were to be prospectively enrolled according to a systematic sampling scheme that allowed a random sampling of patients at each institution among all patients undergoing surgery at that institution.

Of the 5,436 patients enrolled, 5,065 patients completed the study and were included in the final analysis. Of the 371 patients excluded, 32 were excluded due to patient withdrawal, 2 due to death prior to surgery, 97 due to cancellation or rescheduling of surgery, 132 patients due to change in procedure, 11 due to inadvertent enrollment in another study, 86 due to incomplete data and 11 due to incomplete blood sampling, shipping or storage.

Aspirin was administered in doses of 160 mg to 650 mg to 3,001 patients within 48 hours of revascularization. All potential side-effects associated with aspirin use were recorded daily by blinded investigators. Independent investigators coded all medications received-including pro- and anti-thrombotic and pro- and anti-coagulant medications, and blood products—by day throughout hospitalization, as well as at admission and at discharge, or until death.

6.1.2 Study Data

For each enrolled patient, approximately 7,500 fields of data were collected throughout the patient's index hospitalization, from admission until discharge, by independent investigators; treated physicians were blinded to all research data. Data included demographic, historical, clinical, laboratory, eloectrocardiographic, specialized testing, resource utilization, and adverse outcomes. Following last patient enrollment, all data fields for each patient were queried centrally for completeness and accuracy, with all changes documented prior to database closure.

6.1.3 Outcome Measurements

All outcomes were prespecified, defined by protocol, and discerned by investigators blinded to treatment group. Fatal and non-fatal outcomes were classified as cardiac (myocardial infarction, congestive heart failure and cardiac death), cerebral (stroke, encephalopathy and cerebral death), renal (dysfunction, failure and renal death), gastrointestinal (ischemia, infarction and GI death), or other (such as infectious, pulmonary). The diagnosis of myocardial infarction required either: the development of new Q waves (as defined by Minnesota Code 1-1-1I or 1I-2-7); or new persistent S T-segment or T-wave changes (Minnesota Code 4-1,4-2, 5-1 or 5-2) associated with an elevation of CK-MB isoenzyme values; or autopsy evidence of acute myocardial infarction. The diagnosis of heart failure required either: the use of a ventricular assist device; or the use of continuous inotropic support for at least 24 hours; or autopsy evidence of heart failure. Cerebral outcomes were classified as: clinically diagnosed stroke or encephalopathy; or CT, MRI or autopsy evidence of a focal or global defect. Renal dysfunction was defined as: a serum creatinine ≧177 μmol/L accompanied by a ≧62 μmol/L rise over baseline; and renal failure was defined as dysfunction requiring dialysis, or autopsy evidence of renal failure. Gastrointestinal ischemia was defined as abdominal pain diagnosed as intestinal ischemia, or detected at exploration; infarction required bowel resection, or autopsy evidence of intestinal infarction.

6.1.4 Statistical Analysis

The risk of death for aspirin taking versus control populations were compared using the Chi-Square test. Individual ischemic outcomes involving the heart, brain, kidney and gastrointestinal tract, and combined ischemic outcomes were compared using Fisher's Exact Test or the Chi-Square test as appropriate. Odds ratios and the 95% confidence intervals are presented with associated P values. All predictor variables significant at two-tailed nominal P values <0.15 in univariate analyses were then entered into a multivariate logistic model. Stepwise logistic regress was performed, retaining variables significant at two-tailed nominal P values <0.05. All statistical analysis were performed with SAS Version 8.12 software. (SAS Institute, Cary, N.C.)

6.2 Results

Small differences existed between study groups, notably patients receiving aspirin were more likely to have unstable angina, prior PTCA and be treated with beta-blockers, calcium channel blockers and antiplatelet therapy and less likely to have a history of heart failure and be treated with ACE inhibitor (Table 1). No other important differences existed for any medical or surgical characteristic. Most cardiac medications were continued up to the time of surgery, however, anti-platelet medications were discontinued prior to surgery in 50% of patients that had been receiving anti-platelet treatment at hospital admission.

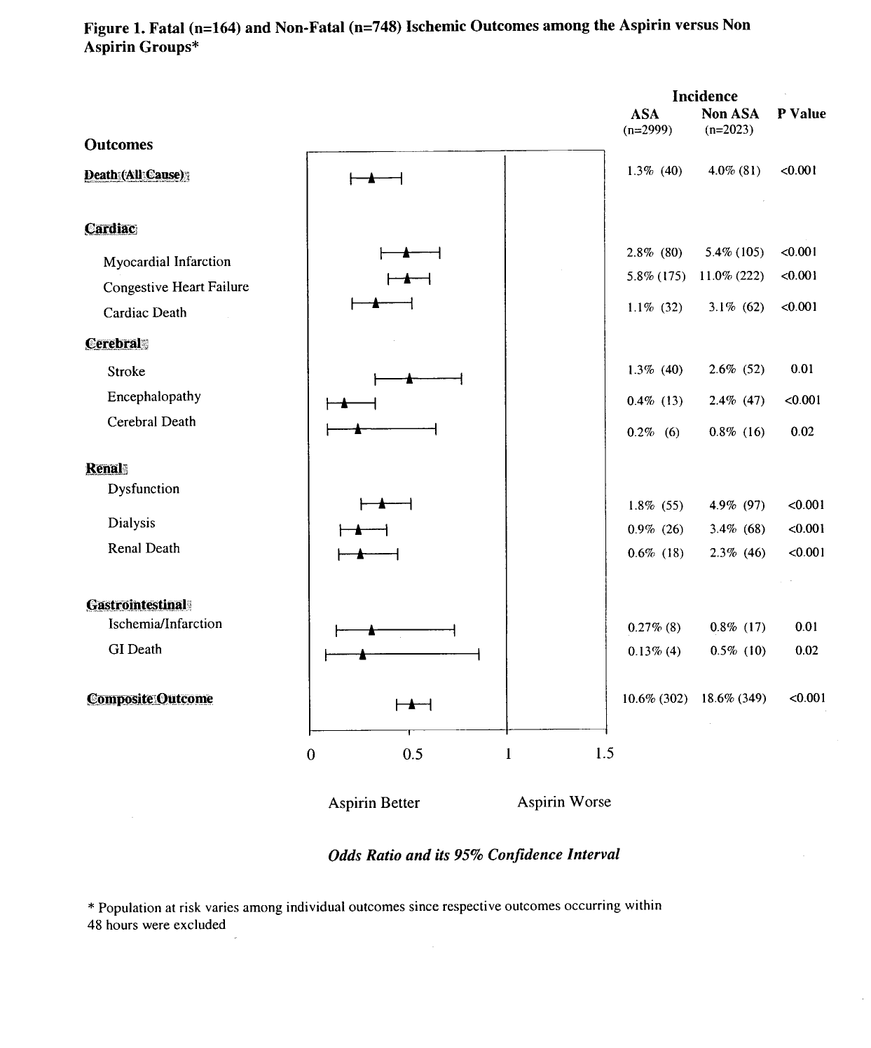

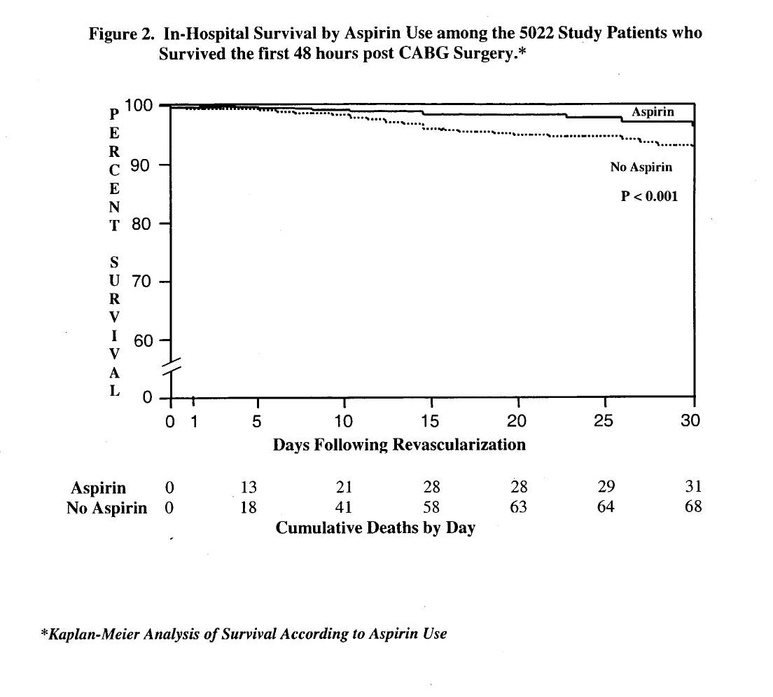

Patients receiving aspirin within 48 hours of revascularization had one-fourth the risk of dying during hospitalization (1.4% v. 5.9%; P<0.0001). Patients receiving aspirin also had one-half the risk of non-fatal ischemic complications associated with the heart, brain kidney or gastrointestinal tract (13.6% v. 24.5%; P<0.0001). FIG. 1. Of those receiving aspirin, none died within 12 hours of surgery (versus 25 patients in the control group) and one died within 48 hours of surgery (versus 42 patients from the control group). FIG. 2.

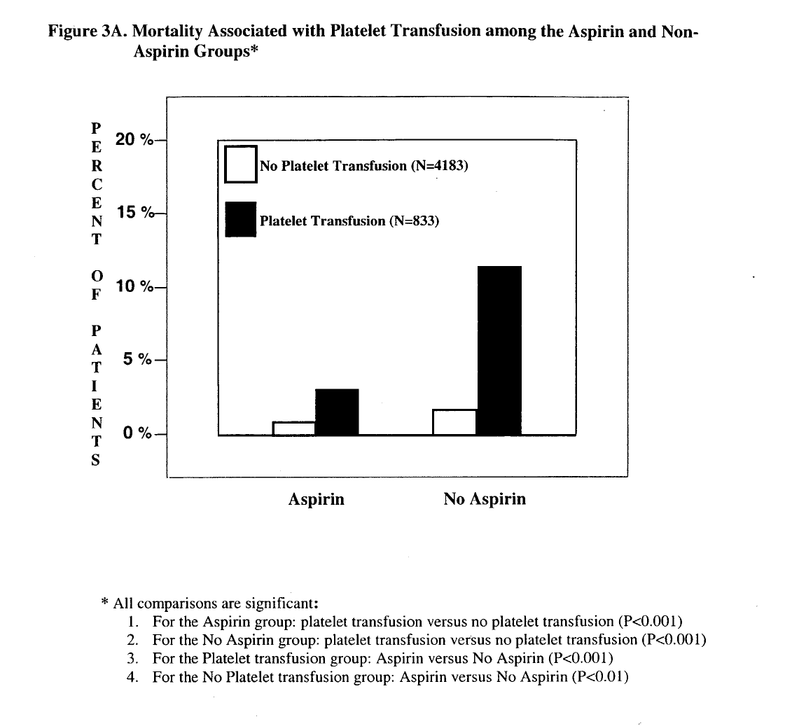

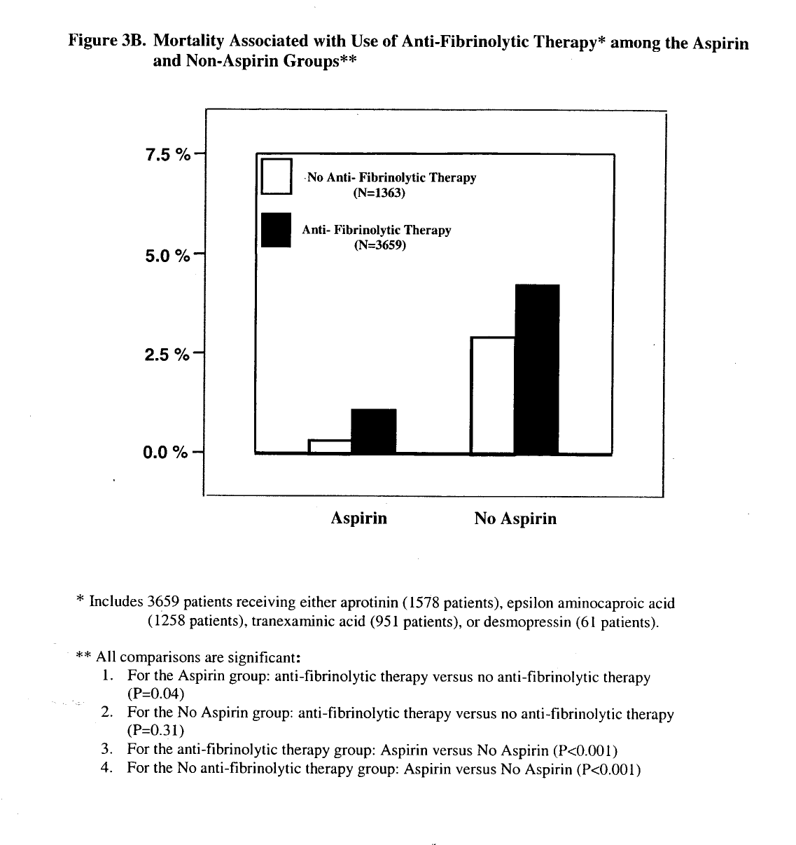

Improved survival over the first 30 postsurgical days was associated only with early aspirin use, as opposed to other reversible factors. (FIG. 2). A first use of aspirin 48 hours after surgery was not associated with a significant reduction in mortality (15%; P=0.534). The beneficial effect of aspirin on fatal outcomes was significant over all subsets including gender, age, geographical region and type of insurance. Length of hospitalization was decreased in those receiving aspirin (9.57±7.14 versus 11.32±9.44; P<0.0001). The risks associated with platelet transfusion after reperfusion and prophylactic anti-fibrinolytics were associated with increased risk of dying and ischemic complications. Aspirin use substantially reduced, but did not eliminate these risks. FIG. 3A. In addition to the unexpected benefits of aspirin use according to the methods, aspirin use was also safe. Table 2.

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below.

All references cited herein are hereby incorporated by reference in their entireties.

TABLE 1
Baseline Demographic and Medical Characteristics Among the 5065 Study Patients

| | | | | Non-Aspirin | |
|---|---|---|---|---|---|
| | | All Patients(N = 5065) | Aspirin Group(N = 3001) | Group(N = 2064) | P Value |
| Age (years) | | | | | |
| | Mean (±SD) | 64.1 (±9.76) | 63.6 (±9.71) | 64.7 (±9.8) | <0.0001 |
| | Median | 64.8 | 64.3 | 65.5 | <0.0001 |
| Body Surface Area < 1.93² | | 49.2% (2478) | 52.6% (1573) | 44.0% (905) | <0.001 |
| Female Gender | | 20.5% (1038) | 19.3% (579) | 22.2% (459) | 0.0107 |
| Race* | | 8.2% (413) | 7.4% (221) | 9.3% (192) | 0.0141 |
| Cardiac History | | | | | |
| Diabetes | | 30.1% (1525) | 29.2% (877) | 31.4% (648) | 0.0941 |
| Hypertension | | 67.3% (3407) | 66.6% (1993) | 68.9% (1414) | 0.0839 |
| Smoking | | 69.2% (3506) | 71.1% (2132) | 66.7% (1374) | 0.0007 |
| Hypercholesterolemia | | 70.6% (3575) | 74.5% (2183) | 69.3% (1392) | <0.0001 |
| Unstable angina | | 50.3% (2550) | 52.1% (1564) | 47.8% (986) | <0.0001 |
| Myocardial infarction | | 51.4% (2603) | 52.5% (1558) | 51.2% (1045) | 0.3498 |
| Congestive heart failure | | 34.7 (1758) | 41.7% (853) | 30.3% (905) | <0.0001 |
| PTCA | | 15.1% (767) | 16.2% (484) | 13.8% (283) | 0.0184 |
| CABG | | 6.0% (306) | 5.9% (177) | 6.3% (129) | 0.6054 |
| Medications | | | | | |
| At hospital admission/prior | | | | | |
| to surgery | | | | | |
| ACE inhibitor | | 40.9%/38.0% | 38.0%/34.9% | 45.0%/42.5% | <0.0001 |
| Beta-blockers | | 62.3%/63.9% | 65.6%/67.9% | 57.6%/58.1% | <0.0001 |
| Calcium channel blockers | | 33.4%/31.8% | 35.7%/33.9% | 30.1%/28.7% | <0.0001 |
| Anti-platelet therapy | | 48.9%/23.8% | 53.4%/26.8% | 42.1%/19.2% | <0.0001 |
| Aspirin | | 46.8%/22.4% | 52.0%/25.9% | 39.2%/17.3% | <0.0001 |
| Dipyridamole | | 0.4%/0/2% | 0.3%/0.1% | 0.5%/0.3% | 0.1938/0.1133 |
| Other | | 4.0%/2.4% | 3.1%/1.8% | 5.2%/3.2% | <0.0001/0.0013 |

*Includes patients with the stated race of African American, American Indian or Hispanic

TABLE 2
Aspirin Use and Adverse Safety Events Among 5065 Study Patients

| | | No aspirin % | |
|---|---|---|---|
| EVENT | Aspirin % (N) | (N) | P value |
| Hemorrhage | | | |
| Gastrointestinal tract bleeding | 1.1% (34) | 2.0% (42) | 0.0099 |
| Other source bleeding | 1.7% (50) | 3.4% (70) | <0.001 |
| Return to operating room for | 1.9% (57) | 5.3% (109) | <0.0001 |
| bleeding | | | |
| Gastritis | 0.33% (10) | 0.15% (3) | 0.1941 |
| Infection | 8.4% (253) | 12.8% (265) | <0.0001 |
| Impaired Wound Healing | 4.5% (134) | 4.5% (92) | >0.9813 |

TABLE 3A
Multivariate Analyses for Mortality

| | | 95% | |
|---|---|---|---|
| | Odds | Confidence | |
| Variable | Ratio | Interval | P value |
| Aspirin use following revascularization | 0.27 | 0.19-0.40 | <0.0001 |
| Previous CABG | 3.07 | 1.91-4.93 | <0.0001 |
| Creatinine > 1.3 μmol/L (admission) | 2.76 | 1.94-3.92 | <0.0001 |
| Prior hospitalization for heart failure | 2.14 | 1.42-3.22 | <0.001 |
| Heart failure (admission) | 1.99 | 1.40-2.83 | <0.001 |
| Unstable angina (admission) | 1.71 | 1.20-2.43 | 0.003 |
| Race* | 2.02 | 1.24-3.29 | 0.005 |
| Warfarin/coumadin** | 2.00 | 1.23-3.25 | 0.005 |
| Heart block (admission) | 1.57 | 1.06-2.32 | 0.025 |
| Age > 70 years | 1.48 | 1.05-2.09 | 0.026 |
| BSA < 1.93 m² | 1.46 | 1.02-2.07 | 0.038 |

*Includes African-American, American Indian or Hispanic.
**Over the week prior to revascularization.

TABLE 3B
Multivariate Analyses for Mortality

| | | 95% | |
|---|---|---|---|
| | | Confidence | |
| Variable | Odds Ratio | Interval | P value |
| Aspirin use (admission) | 1.46 | 0.41-5.15 | 0.557 |
| Aspirin use (prior to surgery) | 0.77 | 1.23-2.64 | 0.681 |
| Discontinuation of aspirin use | 1.04 | 0.28-3.91 | 0.949 |
| Anti-platelet use (admission) | 1.35 | 0.13-14.01 | 0.800 |
| Anti-platelet use (prior to surgery) | 0.74 | 0.09-6.04 | 0.780 |
| Discontinuation of anti-platelet use | 1.00 | 0.08-12.25 | 0.999 |

1. A method to prevent post-surgical related morbidity and mortality, in a patient comprising administering within 12 hours after a cardiac surgery to said patient an effective amount of a blood clotting inhibitor, wherein said cardiac surgery does not include vein graft coronary artery bypass graft (CABG) surgery.

2. (canceled)

3. (canceled)

4. (canceled)

5. (canceled)

6. (canceled)

7. (canceled)

8. The method of claim 1 wherein the blood clotting inhibitor is administered within 1 hour after the cardiac surgery.

9. (canceled)

10. The method of claim 6 wherein the blood clotting inhibitor is administered daily for 10 days after the cardiac surgery.

11. (canceled)

12. (canceled)

13. (canceled)

14. (canceled)

15. (canceled)

16. The method of claim 40 wherein the non-cardiac surgery is abdominal surgery, neurological surgery, gynecological surgery, orthopedic surgery, urological surgery, vascular surgery, or surgery related to otolaryngology.

17. The method of claim 1 wherein the blood clotting inhibitor is selected from a group consisting of an antiplatelet agent, thrombolytic enzyme, aggregation inhibitor, glycoprotein IIb/IIIa inhibitor, glycosaminoglycan, thrombin inhibitor, anticoagulant, heparin, low molecular weight heparin, coumarin, indandione derivative, and tissue plasminogen activator.

18. The method of claim 1 wherein the blood clotting inhibitor is administered orally.

19. The method of claim 1 wherein the blood clotting inhibitor is administered parenterally.

20. The method of claim 17 wherein the blood clotting inhibitor is an antiplatelet agent.

21. The method of claim 20 wherein the antiplatelet agent is aspirin.

22. The method of claim 21 wherein the oral dose is 40 mg/kg to 5 gm/kg.

23. The method of claim 20 wherein the antiplatelet agent is dipyridamole.

24. The method of claim 23, wherein the oral dose is 5 mg/kg to 500 mg/kg.

25. The method of claim 23, wherein the intravenous dose is 0.1 mg/kg to 1 mg/kg.

26. The method of claim 20 wherein the antiplatelet agent is a combination of dipyridamole and aspirin.

27-30. (canceled)

31. The method of claim 6 wherein said blood clotting inhibitor is administered at least once daily for 6 months after the cardiac surgery.

32. The method of claim 6 wherein said blood clotting inhibitor is administered at least once daily for one year after the cardiac surgery.

33. The method of claim 1 wherein said post-surgical related morbidity and mortality comprises myocardial infarction, congestive heart failure, serious cardiac dysrhythmia, or stroke.

34. The method of claim 1 wherein said post-surgical related morbidity and mortality comprises encephalopathy, cognitive dysfunction, or cerebral death.

35. The method of claim 1 wherein said post-surgical related morbidity and mortality comprises renal dysfunction, renal failure, or renal death.

36. The method of claim 1 wherein said post-surgical related morbidity and mortality comprises gastrointestinal infarction, ischemia, or death.

37-39. (canceled)

40. A method to prevent post-surgical related morbidity and mortality in a patient undergoing a non-cardiac surgery, said method comprising administering to said patient an effective amount of a blood clotting inhibitor within 48 hours after said non-cardiac surgery.

41. The method of claim 40 wherein the blood clotting inhibitor is selected from a group consisting of an antiplatelet agent, thrombolytic enzyme, aggregation inhibitor, glycoprotein IIb/IIIa inhibitor, glycosaminoglycan, thrombin inhibitor, anticoagulant, heparin, low molecular weight heparin, coumarin, indandione derivative, and tissue plasminogen activator.
